(19) United States
(12) Patent Application Publication
(10) Pub. No.: US 2012/0264198 A1
(43) Pub. Date: Oct. 18, 2012

(54) In-Situ Reclaimable Anaerobic Composter

(75) Inventors: Gary R. Hater, Cincinnati, OH (US); Roger B. Green, Cincinnati, OH (US); Christopher J. Pierce, Franklin, IN (US); Kevin Mieczkowski, Goshen, KY (US); Gregory C. Cekander, The Woodlands, TX (US)

(73) Assignee: WASTE MANAGEMENT, INC., Houston, TX (US)

(21) Appl. No.: 13/399,302

(22) Filed: Feb. 17, 2012

Related U.S. Application Data
(62) Division of application No. 12/706,385, filed on Feb. 16, 2010.
(60) Provisional application No. 61/152,867, filed on Feb. 16, 2009.

(51) Int. Cl. C12M 1/107; C12S 3/00

(52) U.S. Cl. 435/267

(57) ABSTRACT

An in-situ dry anaerobic composter containing 40% to 75% by weight solids and located in a section of ground including a pit having side walls and a bottom, an essentially impervious liner located in the pit such that the liner abuts the pit side walls and bottom to form a lined pit, a compostable material located in the lined pit and a gas management system for extracting a gaseous anaerobic decomposition product from the compostable material as well as methods for operating the anaerobic composter.

This application is a divisional of U.S. application Ser. No. 12/706,384 filed on Feb. 16, 2010, which claims priority to provisional application Ser. No. 61/152,867, filed on Feb. 16, 2009, the specifications of which are incorporated by reference in their entirety.

### BACKGROUND OF THE INVENTION

(1) Field of the Invention

This invention concerns in-situ dry anaerobic composters as well as methods for their construction and operation.

(2) Description of the Art

The European community has been using anaerobic digesters to remediate food and yardwaste for many years. Manufacturers like Becon, Drainco, and Kompogas have been successfully building and operating these units in Europe and Asia for a number of years. An example of a prior art composter/digester is shown in FIG. 1 where the digester 100 includes a pile of compostable material 102 that lies on a clay liner base 104. The compostable material 102 is covered by a geomembrane cap 105 which, in turn, is covered with an optional insulating layer 106 such as cellulose. Between the clay liner base 104 and the compostable material 102 lies leachate extraction piping 108 and gas extraction piping 110. Within the compostable material 102 lies lechate recirculation piping 112. Finally, a soil berm 114 surrounds the digester.

Disposal and recycling fees in countries where anerobic digesters are used are supported by a tax base that makes their construction and operation affordable. Capital cost for these dry anaerobic digesters are typically $300 to $500 per ton of capacity. For example a 24,000 tons per year facility costs between $8,000,000 and $13,000,000. This capital cost leads to an amortization cost per ton for a 20 year life of site plant of about $20 to $40 per ton in today's market which is too high to be economically feasible in the United States. There is a need, therefore, for reusable anaerobic digesters that have been improved in a manner that causes them to be economically feasible in the United States and more profitable when used outside of the United States.

### SUMMARY OF THE INVENTION

The present inventions demonstrate at least one of the following advantages. The present invention is directed to in-situ and reusable anaerobic digesters (composters) with capital costs that are up to 60% to 80% lower than prior art anaerobic digesters while providing similar or better gas yields per ton. It is believed that the digesters of the present invention are economically feasible in the U.S. and Canada.

Another aspect of the present invention is a flexible anaerobic digester complex that allows for the construction of different sized digester cells depending upon the anticipated dispersion of heat that will be generated during the fermentation process. The complex will include many small digester cells in warmer weather locations where fermentation heat is not easily dispersed and larger digester cells in cooler weather locations.

Still another aspect of the present invention are anaerobic digesters that allow for a decrease the parasitic heating load by placing it in-situ and by providing for indirect heating or warming of the fermenting mass.

In a further aspect, the present invention includes an in-situ dry anaerobic composter comprising a section of ground including a pit having side walls and a bottom; an essentially impervious liner located in the pit such that the liner abuts the pit side walls and bottom to form a lined pit; a compostable material located in the lined pit; a gas management system for extracting a gaseous anaerobic decomposition product from the compostable material; at least one pipe for injecting an aqueous stream into the compostable material; and at least one pipe for removing aqueous materials that collect on the bottom of the lined pit from the composter.

Yet, another aspect of the present invention is a method for composting material in a in-situ reusable dry anaerobic composter cell, the method including the steps of; preparing compostable material for fermentation; preparing a cell for holding the compostable material the cell including a pit constructed in a section of ground, the pit including side walls, a bottom, an essentially impervious liner located in the pit such that the liner abuts the pit side walls and bottom to form a lined pit; placing the prepared compostable material in the cell; covering the cell with a cover to form an essentially gas tight anaerobic composter cell; bringing the cell to fermentation conditions and operating the cell at anaerobic fermentation conditions sufficient to form digestate and anaerobic fermentation gasses; collecting the anaerobic fermentation gasses using gas extraction piping located in the cell; halting the anaerobic fermentation when a defined anaerobic fermentation end point is reached; and opening the cell and removing the digestate to form an emptied cell.

### DESCRIPTION OF THE FIGURES

FIG. 1 is a cross-section view of a prior art digester/composter. In the prior art digester, fermentation product gas is removed from the bottom of the bioreactor and leachate is added to the fermentation zone at various levels above the ground;

FIGS. 2A and 2B are plan and section views of in-situ reclaimable anaerobic composter cell (RAC cell) embodiments of this invention. The RAC cell includes a pit 20 excavated in the ground. Pit 20 includes walls 22 that are covered with an essentially impermeable liner 24 such as a HDPE liner. Pit 20 and liner 24 can be reused multiple times. The composter can be constructed at a variety of locations such as in a landfill lift, in the open ground, in a covered structure or at any location where the composter is needed or can be constructed;

FIG. 3 is a plan view of a plurality of in-situ RAC cells 10 where each of the plurality of RAC cells is associated with one or more of the same leachate circulation system 12, the same gas management system 14, the same vacuum extraction system 16, and the same bio-filter 18;

FIGS. 4A and 4B are a plan and section views of an in-situ RAC cell embodiment if this invention including additional details of composter features;

FIG. 5 is a partial cross-section view of an embodiment of a top edge of an in-situ RAC cell 10 showing piping exiting the cell through a soil plug 26 and piping penetration plate 28;

FIGS. 6A and 6B are top and side views of a piping vault 42 useful in RAC cells 10 of the present invention;

FIGS. 7A and 7B are plan and section views of an in-situ RAC cell showing an optional gas extraction piping configuration embodiment;

FIGS. 8A and 8B are plan and section views of an in-situ RAC cell embodiment showing a vacuum extraction piping and bio-filter system embodiment of this invention;

FIGS. 9A, 9B and 9C are plan views of in-situ RAC cell embodiments of this invention including several geomembrane cap embodiments;

FIG. 10 is a close-up side cutaway view of an edge of a RAC cell 10 that includes a piping penetration vault 42.

FIG. 11A is a cross-section view of an in-situ RAC cell embodiment of this invention and FIG. 11B is a close-up cross section view of an anchor trench associated with the composter of FIG. 11A; and

FIGS. 12A and 12B are plan and section views of yet another in-situ RAC cell embodiment of this invention.

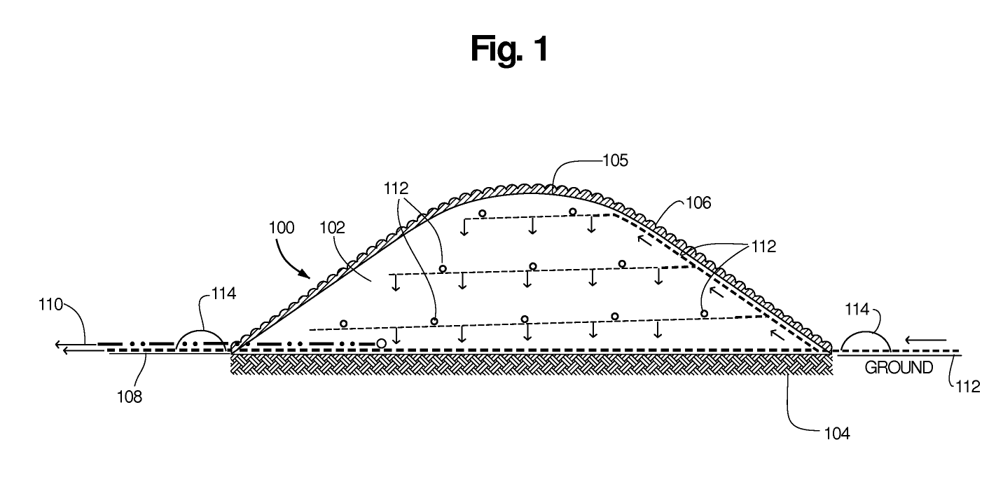

### DESCRIPTION OF THE INVENTION

The present invention relates to an improved organics diversion system that includes one or more batch in-situ reusable anaerobic composter cells—RAC cells 10. The RAC cells 10 of this invention use flexible membrane liners as construction materials and accept and remediate shredded compostable materials. The RAC cells 10 can be used to compost any type of compostable material know in the art including, but not limited to, yard waste, manure, sludges, wood, pallets, brush, food waste, cellulosic materials like cardboard, construction waste, and combinations there of. RAC cells 10 are typically operated in a manner that produces both methane for energy and useful solid. The solids that are not fermented to form methane gas are reclaimable as digestate or compost solids. The resultant solids are useful as soil amendment, as a peat moss substitute or as compost.

In one embodiment, the RAC cells 10 of this invention are used to compost a mixture of yard waste and food waste in a dry fermentation (50% to 70% solids) process. The RAC cells 10 of this invention may be arranged in an array of two or more RAC cells to form a composting complex. Each individual RAC cell 10 is generally operated as a discrete batch. Cycle time is variable and is dependent on feedstock methane potential and weather. Anerobic cycle time can vary from about 30 days to several months or more.

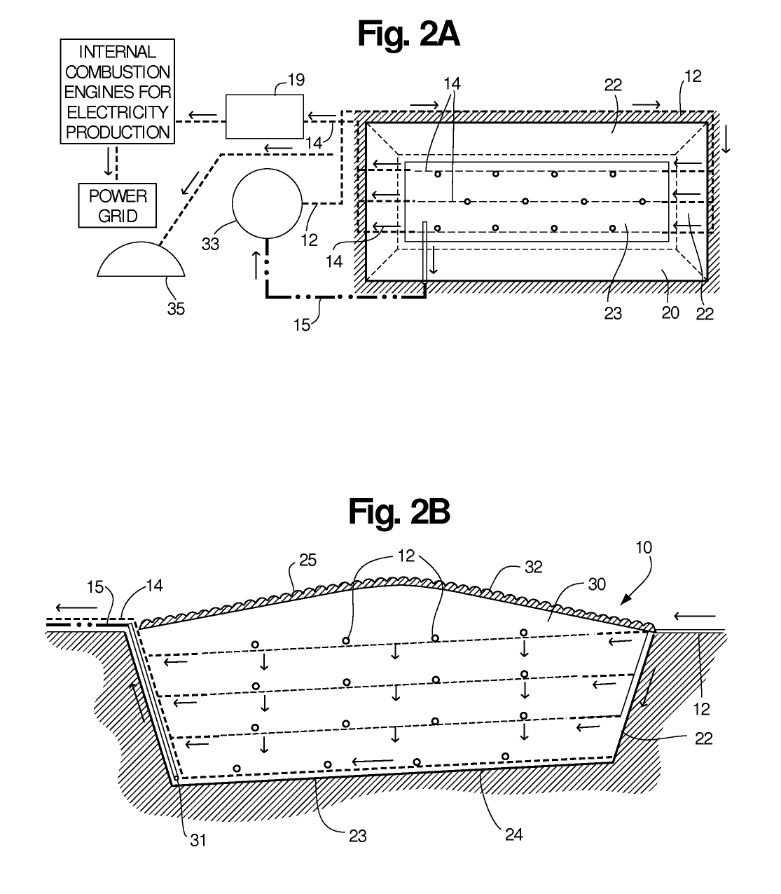

Further details of this invention are presented below, in part by reference to the accompanying Figures. Referring now to FIGS. 2A and 2B there are shown a plan and side cross section views of an in-situ RAC cell 10 of this invention. RAC cell 10 is located in a pit 20 constructed in the ground. Pit 20 includes walls 22 and a bottom 23. A liner 24 covers walls 22 and bottom 23. In addition, a liner cover 25 covers the top of compostable material 30 located in pit 20 thereby forming an essentially gas tight seal around pit 20 and compostable material 30. Optional cover material 32, such as a fiberglass cap, a second liner cover on top of liner cover 25, a liner cover 25 filled with air, sliding panels, sheets of foam board, cellulose, combinations thereof and any other useful insulating materials be applied over or under cover 25 to aid in RAC cell heart retention. In another embodiment, cover material 32 can be a biofilter material such as wood chips including microorganisms that consume odor compounds and other components of the anaerobic fermentation gases that might seep from RAC cell 10.

Pit 20 can be constructed by any conventional methods such as by using a bulldozer or an excavator. The walls 22 and/or bottom 23 of pit 20 will typically be formed of soil. However, the walls can, if desired, be formed of structural materials such as concrete or pilings driven into the ground.

RAC cell 10 will have a width of about 50 feet but can be from about 30 inches to 70 feet wide. The cell will have a depth of from about 6 inches up to a depth of about 20 feet. The RAC cell length will generally be between 40 feet and 300 feet with a more typical length ranging from about 80 feet to about 120 feet in length. The apex of RAC cell 10—which typically lies above grade—allows for a 2% to 10% slope (preferably about 4%) on the top of the cell. Pit wall slopes are typically 1.5/1 or steeper, up 0/1 (or vertical). In some cases the end wall 22′ associated with leachate recirculation piping can be constructed with a gentler angle of from about 3/1 to 4/1 to allow the digestate (the RAC cell product) to be removed by a loader or dozer during the removing step.

RAC cell 10 shown in FIGS. 2A and 2B further includes leachate injection piping 12, gas extraction piping 14 and leachate extraction piping 15. Leachate injection piping 12 is orientated in compostable material 30 such that leachate is injected into the compostable material at several different vertical points. Moreover, leachate injection piping 12 is preferably constructed to include perforations or outlets that allow leachate or any other source of water to be dispersed as evenly as possible throughout compostable material 30. Similarly, gas extraction piping 14—which also includes perforations or openings within RAC cell 10—is positioned in the RAC cell to remove gas generated during anaerobic fermentation of the cell mass. Finally, RAC cell 10 includes at least one sump pit 31 preferably placed at a low point in RAC cell 10. Any leachate formed in RAC cell 10 collects in sump pit 31 where a sump pump including an inlet in the sump pit removes the collected leachate from RAC cell 10. The sump pump moves leachate through leachate removal piping 15 where it can be directed, for example, to leachate recirculation system 33 for recycling back into leachate injection piping 12, it can be directed to a storage tank or it can be directed to both locations simultaneously.

A unit operation that is typically shaped by two or more RAC cells 10 is a biofilter 35. Biofilter 35 can be any type of structure or device that is able to safe fully and effectively remove unwanted materials such as volatile organic compounds, methane, and sulfur compounds from gases collected in the fermentation mass and headspace in RAC cell 10 that would otherwise cause unwanted odors and/or emissions. An example of a useful biofilter is a trench including wood chips that have been seeded with or that includes microorganisms that remediate the odor compounds and other organic compounds in gases withdrawn from the RAC cell. The RAC cell gases are directed to the bottom of the biofilter and allowed to percolate through the biofilter into the atmosphere.

The gas extracted from RAC cell 10 by gas extraction piping 14 is directed to gas management system 19. The anaerobic fermentation gases will typically be rich in methane and carbon dioxide and will include smaller amounts of other gases such as ethane, nitrogen, oxygen, and so forth. The gas management system extracts valuable biofuel as methane from the anaerobically fermenting mass which is typically food waste and yard waste. The extracted gases typically include methane in an amount ranging from 50% to 74% by volume. The fermentation gas is preferably extracted by vacuum and is preferably directed to an energy processing facility. In one embodiment, the methane rich gas recovered by gas management system 19 is directed to internal combustion engines for electricity production. Alternatively, the extracted methane rich gas can be used for any purposes that methane is used such as for heating, steam generation or in chemical processes

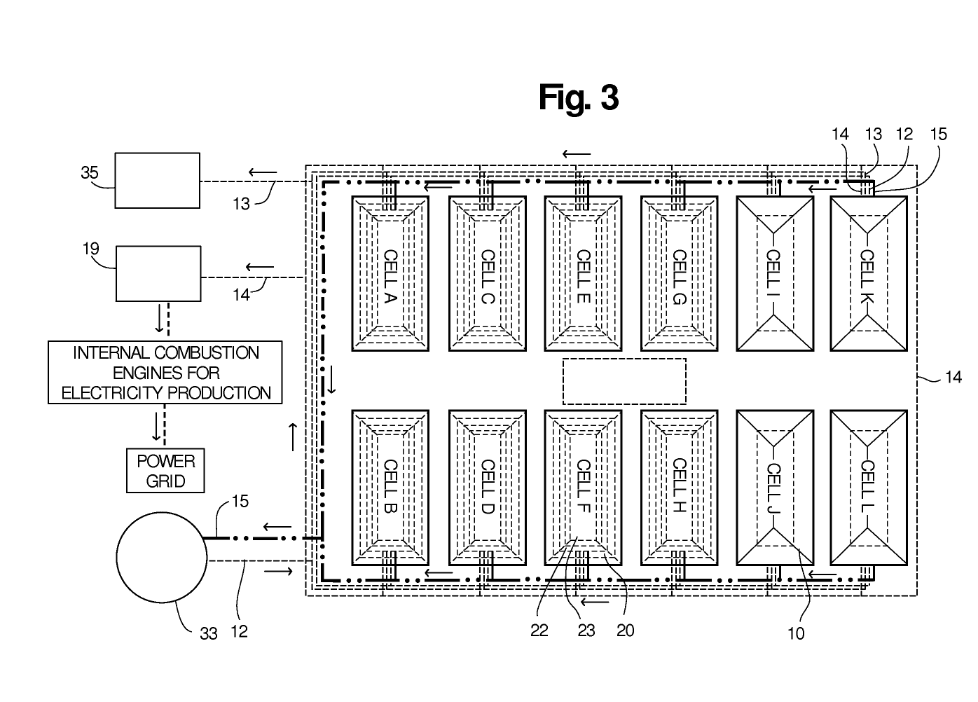

FIG. 3 is plan view of a composter complex including a plurality of RAC cells 10 arranged such that they share leachate injection and withdrawal piping and systems, gas extraction piping and system, vacuum extraction piping and system and other common piping and systems. In FIG. 3, RAC cells A through G show details of pits 20 while cells H through L are covered and operating RAC cells. In FIG. 3, RAC cells A through L from a composter complex that provided for reduction in individual cell operating costs by sharing unit operations such as leachate removal systems and electricity generation systems. Moreover, arranging the RAC cells into a complex allows for the efficient reuse of the RAC cell once the composting process in an individual cell has reached its designated endpoint. The shared unit operations can be kept in operation even when an individual RAC cell is being constructed or renewed by bringing the individual RAC cells on-line to taking them off-line in a time-wise incremental manner.

Note that in FIG. 3, all of the piping is typically routed around the perimeter of the cell system and located in trenches to allow for better gas collection and prevent pipe crushing because of traffic adjacent to cells 10. While the system shown in FIG. 3 is typical, it is possible to place RAC cells 10 on landfill cells in which case the RAC cells are erected adjacent to each other in a long row. When pulling a vacuum to prevent odors as cells are being filled with compostable material, the cells may share a common biofilter 35. When in the fermentation stage all cells share a common gas collection header as well as plumbing for adding liquid or removing liquid from cells.

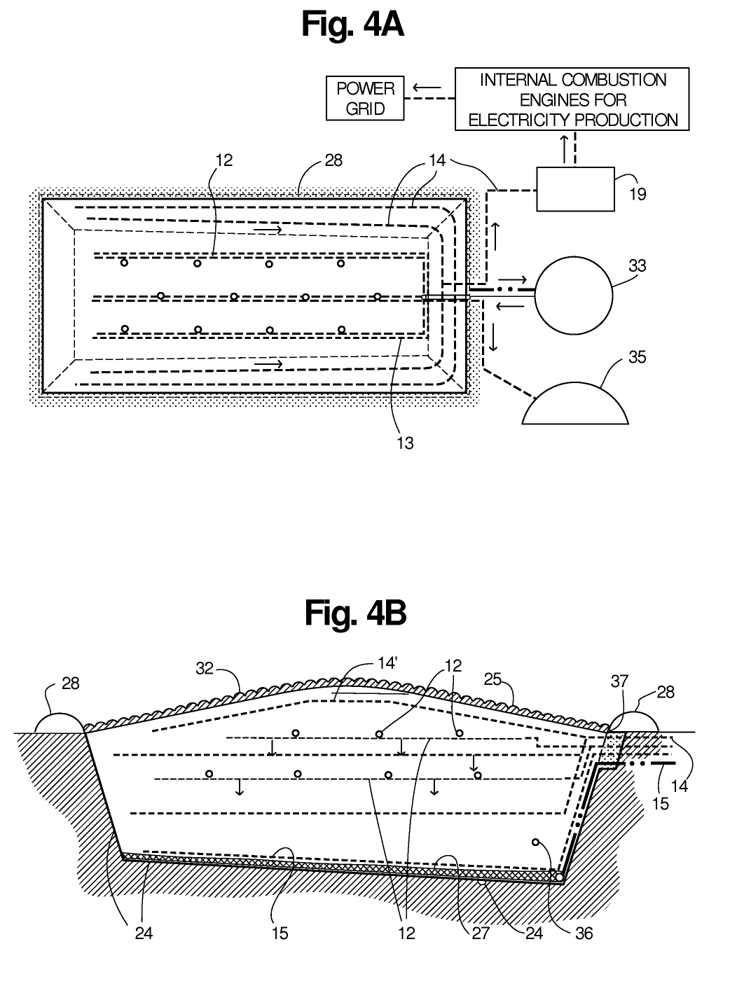

FIGS. 4A and 4B are plan and cross-section views of an in-situ and anaerobic RAC cell 10 of this invention. The RAC cell 10 shown in FIGS. 4A and 4B include a permeable material layer 27 located at bottom of pit 20 between liner 24 and compostable material 30. Permeable materials useful in permeable material layer 27 can be, for example, gravel, sand, tire chips, wood chips and so forth. Preferred permeable materials are wood chips and yard waste because they are compostable and can be removed from RAC cell 10 when the composting process is complete. In another embodiment the bottom of pit 20 includes a liner 24 covered by a geotextile layer 37 which in turn is covered by permeable material layer 27.

As RAC cell 10 is being filled with compostable material, the material can sit in an anoxic state while additional materials are added. This series of additions can take weeks. During that time a vacuum can be intermittently or continuously applied to the partially filled pit that has a temporary cover using aeration piping system 13. The malodors, volatile organic carbon and odor causing sulfur compounds in the extracted gases are directed to biofilter 35 where they are removed form the extracted air biologically.

FIGS. 4A and 4B also show details of piping systems associated with RAC cell 10. The piping systems include gas extraction piping 14, aeration system piping 13, leachate removal piping 15 and leachate injection piping 12. In addition to using aeration system piping 13 to remove gasses from the cell mass during cell construction, aeration system piping 13 also allows air to be blown through the compostable material mass at start up in order to provide an environment in which heterotrophic bacteria consume organic acids and generate heat. Otherwise the organic acids would decrease RAC cell pH and inhibit methane generation. Aeration system piping 13 also allows air to be injected into the RAC cell mass at the end of fermentation to displace residual methane and to begin the compost maturation process.

Other details of significance shown in FIGS. 4A and 4B include a liner 24 to seal the RAC cell and for directing liquid drainage within RAC cell 10 to the sump, sensors 36, a berm 28 such as a soil berm to prevent liquids from running off the RAC cells and piping offset 37 to protect the piping from being crushed during RAC cell excavation.

RAC cell 10 of FIGS. 4A and 4B further include gas extraction piping 14′ associated with a top most portion of RAC cell 10. By “topmost portion”, it is meant that the gas extraction piping 14′ is located from about 0 to about 2 feet from the liner cover 25. In addition, RAC cell 10 optionally includes one or more sensors 36 for monitoring temperature, gas content, redox potential, pH and so forth in RAC cell 10.

Liner 24 and liner cover 25 can be selected from any geomembrane material that is commonly used in landfills. Such geomembrane materials are essentially water and gas impervious. The liners will preferably be selected from a polymer material such as high density polyethylene (HDPE), polyvinyl chloride (PVC) or linear low density polyethylene (LLDPE). The liner thickness will range from about 20 mil to 100 mil or more. In addition, liners 24 and cover 25 can be formed from a combination of layers—both permeable and impermeable so long as at least one layer is essentially gas and liquid impermeable.

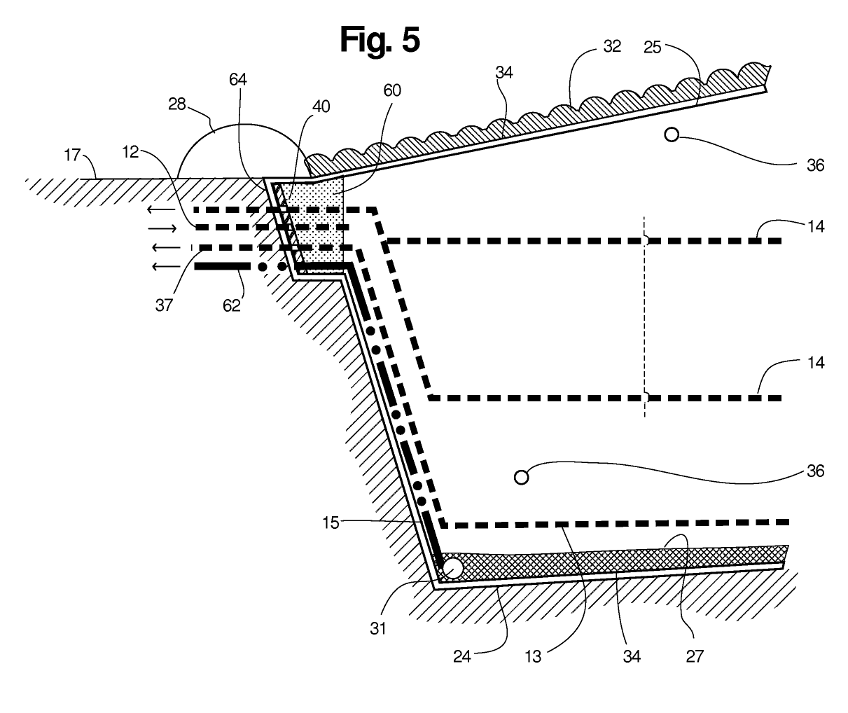

FIG. 5 is a cross-section view of an edge of a RAC cell 10 embodiment of this invention showing a piping offset 37. Piping offset 37 is useful for preventing gases that are formed in RAC cell 10 from uncontrollably escaping and/or entering RAC cell 10 during operation. Piping offset 37 also provide a location where piping can enter and exit RAC cell 10 below grade 17 where the piping is less likely to be damaged during RAC cell erection, operation and turnover. The gases generated during RAC cell 10 fermentation have an unpleasant odor as they include methane, sulfur compounds and other noxious combustible gases. Therefore, preventing the fermentation gases from uncontrollably exiting RAC cell 10 and into the atmosphere is important. Piping offset 37 at least inhibits such unwanted gas migration.

In the embodiment shown in FIG. 5, piping offset 37 is a trench that takes the form of a shoulder 60 formed by a rim 62 and an angled wall 64 that extends away from pit wall 22 at a point where wall 22 meets grade 17. Piping offset 37 is filled with a soil or clay plug 39 and it includes a plate 40 covering angled wall 64. Plate 40 will preferably include apertures through which pipes that direct gases and liquids into and out of RAC cell 10 can pass in a sealed manner. Plate 40 may be made of any material, such as a metal or plastic that is used in landfill and bioreactor construction. It is preferred that plate 40 is made of high density polyethylene.

In order to further seal RAC cell 10 in the region of piping offset, liner 24 preferably covers the rim 62 and angled wall 64 of piping offset 37. Piping offset 37 may be associated with an edge of RAC cell 10 only where piping is entering and exiting the landfill. Alternatively, piping offset 37 may be formed around part to all of the top perimeter of pit 20 to form an anchor trench around the perimeter of cell 10 that, in combination with a soil plug or other seal material anchor liner 24 and cover 25 in place in RAC cell 10.

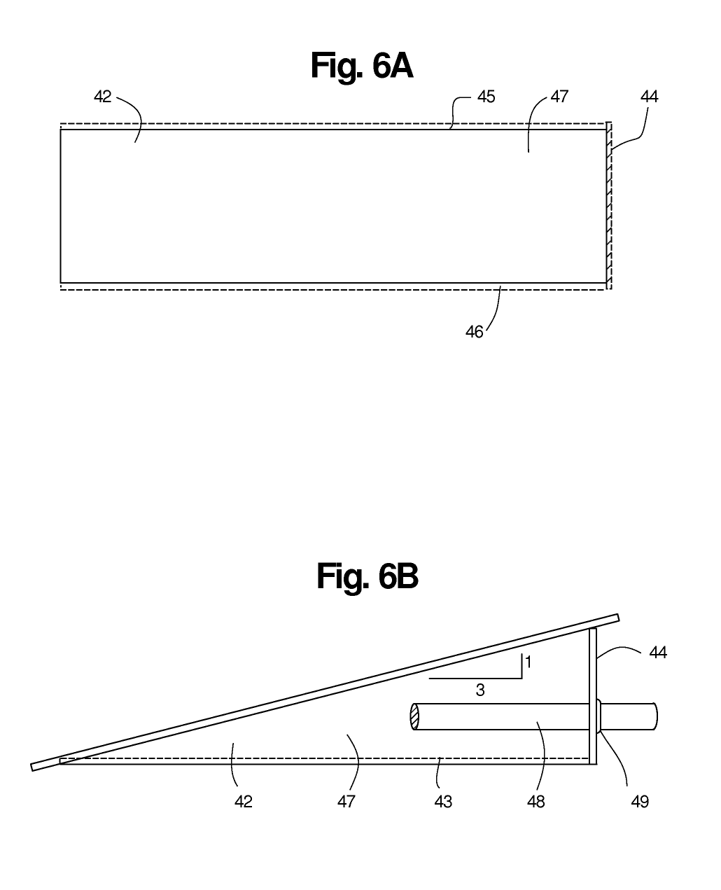

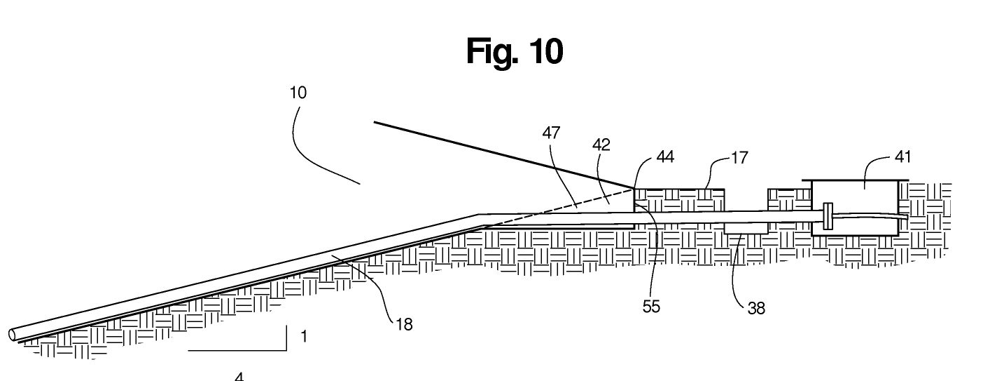

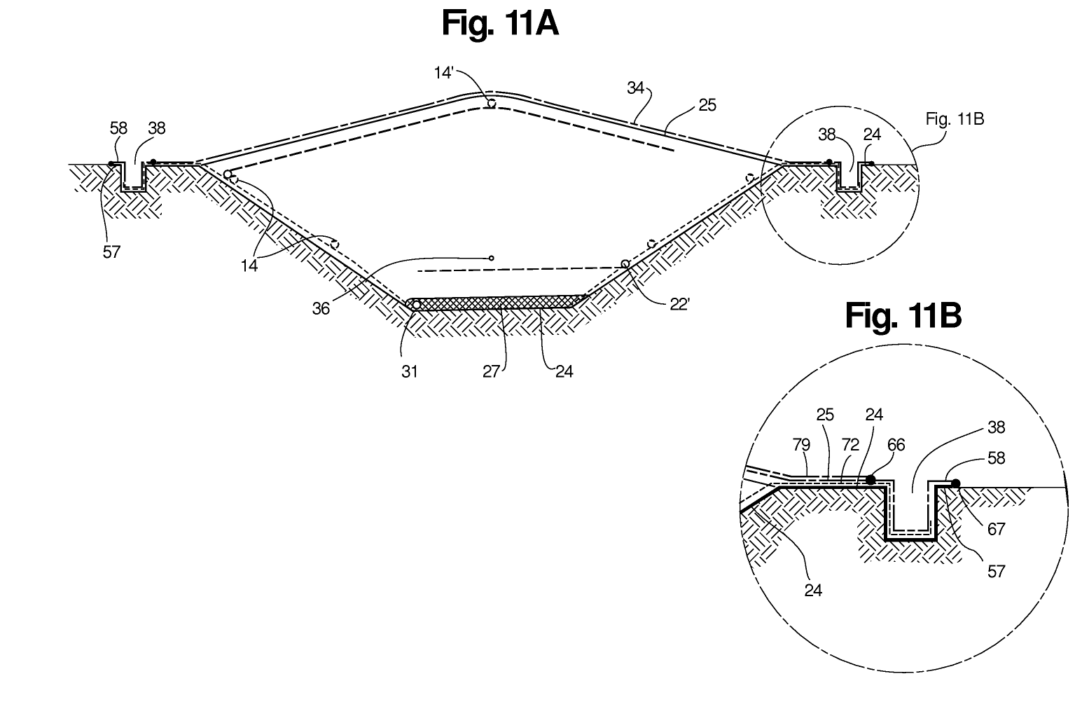

FIGS. 6A and 6B are top and side views of a piping vault 42 that is useful in RAC cells of the present invention. Piping vault 42 will typically be associated with an upper edge of RAC cell 10 as shown, for example in FIG. 10. Piping vault 42 includes a bottom 43, a vertical end wall 44 the combination of which separates angled vertical side walls 45 and 46. The combination of bottom 43, vertical wall 44 and side walls 45 and 46 form a trough 47 through which piping can be directed from and to RAC cell 10.

FIG. 6B shows a pipe entering trough 47 through a vertical wall 44. The pipe includes an extrusion weld 49 at vertical wall 44. Pipe 48 may be one of the pipes associated with one of the piping systems found in RAC cell 10 or pipe 48 may provide a conduit through which one of the pipes associated with the piping system may pass. Piping vault 42 will typically be located, as shown in FIG. 10, at an upper edge of RAC cell 10 such that the top of vertical wall 44 is near, at or above grade 17. Piping vault 42 can be made of any material that is useful in a composter. Useful materials include metals such as galvanized iron or aluminum or plastics such as high density polyethylene or polyvinyl chloride. A preferred piping penetration vault material is high density polyethylene.

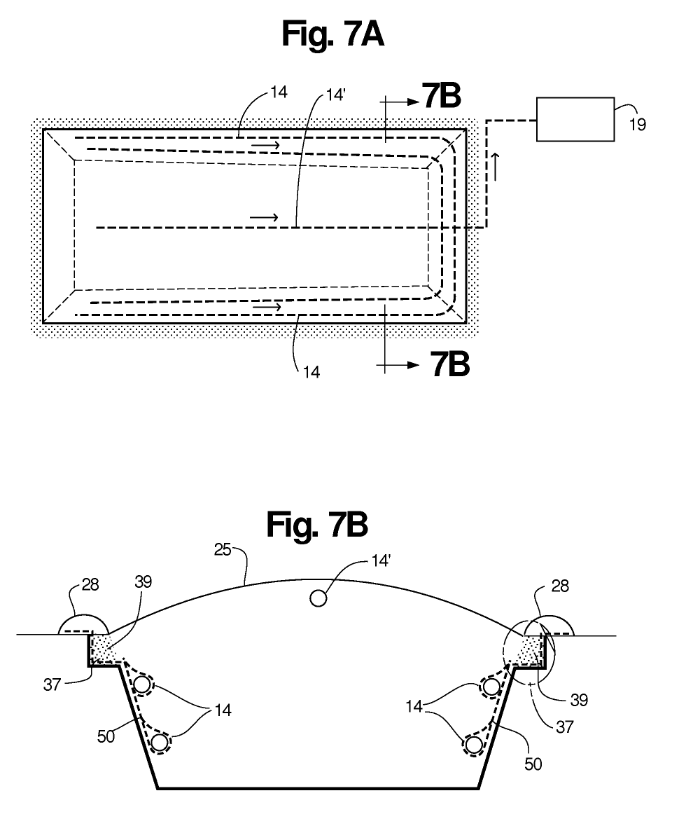

FIGS. 7A and 7B are plan and cross section views of an alternative embodiment for locating gas extraction piping 14 in an RAC cell 10. In FIGS. 7A and 7B, plastic gas extraction piping is wrapped in a geotextile sheet 50 which is suspended from piping offset 37. The geotextile is preferably wrapped within a geotextile material that is permeable to liquid and gas. Geotextile materials include any natural or synthetic fabric material sheets that are highly permeable to liquids and/or gases and that, when used to cover liner 25 and or cover 25 are capable of acting as a barrier to prevent damage to underlying liner layers. Wrapping piping 14 in a sheet of geotextile material allows the piping to be lowered into pit 20 from outside of the pit. In addition, the geotextile sheet 50 is secured into place around the perimeter of pit 20 by directing the edge of the geotextile sheet 50 into piping offset 37 or into an anchor trench 38 and then backfilling with a material such as a plug of soil. The gas extraction pipes 14 thus installed are considered permanent for use in many fermentation cycles for this cell, but are designed for replacement if they are crushed during loading or unloading. The gas collection piping 14′ on the top half of the FIG. 7B, RAC cell 10 is removable.

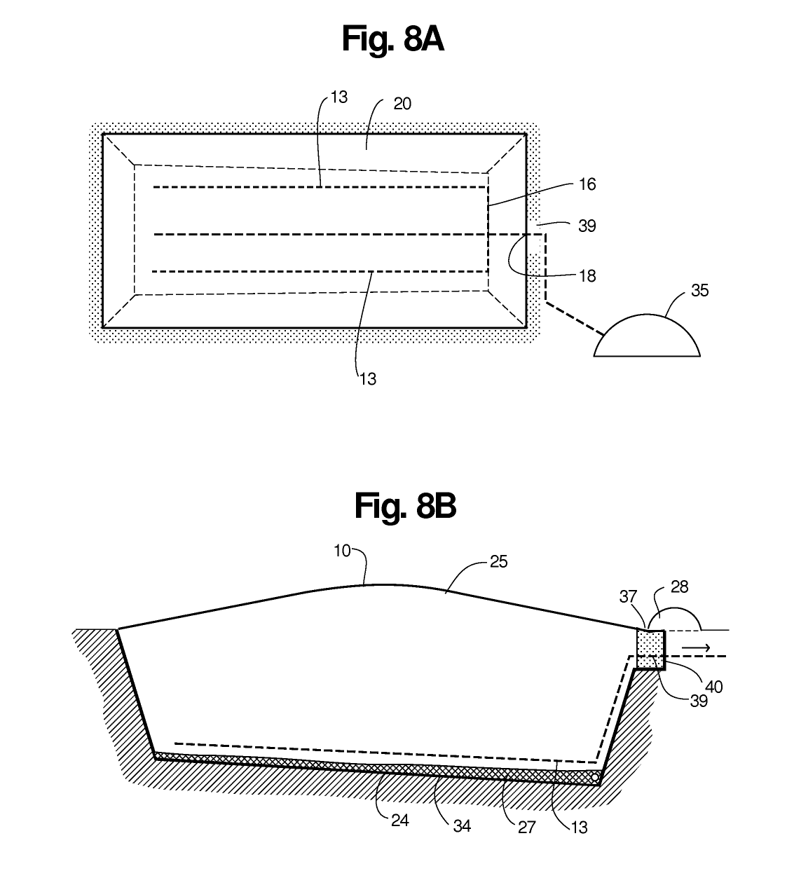

FIGS. 8A and 8B are plan and side cut away views of a RAC cell 10 of this invention including further details of aeration piping 13. The vacuum/aeration piping 13 is used to apply a vacuum during loading of RAC cell 10 and in order to remove air from the RAC cell to quickly bring the RAC cell to anaerobic conditions. Vacuum/aeration piping 13 may also be used for aeration—to direct air into RAC cell 10 prior to opening the reactor when the anaerobic digestion cycle is complete. Piping 13 shown in FIGS. 8A and 8B include a piping manifold 16 located at or near the bottom of pit 20. The manifold is tied into a single exit pipe 18 that is directed through a piping penetration vault 42 or through piping offset 37 where it is directed to a biofilter 35 to remove odor bodies and other undesirable emissions.

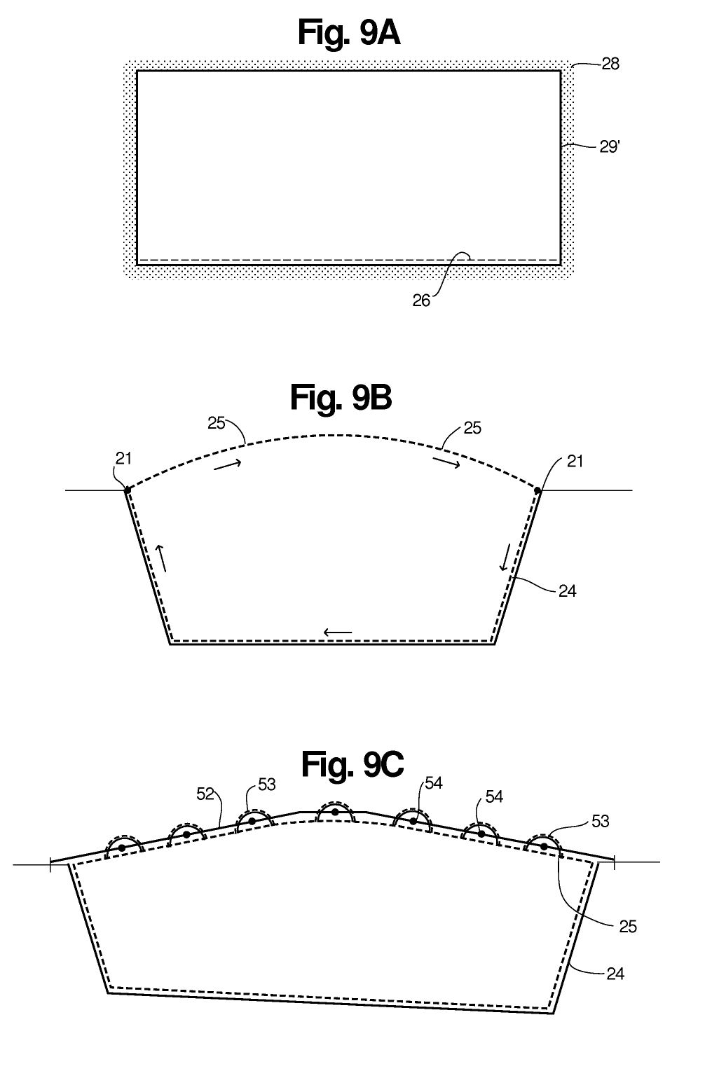

FIGS. 9A, 9B and 9C show various liner and cover configurations useful in RAC cells of the present invention. FIG. 9A shows is a RAC cell including a one piece liner 24′ in which the flexible membrane liner starts on one wall and is welded at weld 26 by lapping the opposing end of the liner at the starting point. FIG. 9B illustrates a two piece flexible membrane liner 24 in which the cover 25 is a separate piece that is welded to liner 24 at all four edges 21. FIG. 9C details a free standing roof 52 on top of a RAC cell 10. Membrane hoops 53 are connected to roof 52 and metal poles 54 are used to externally support the roof 52 by spanning with width or length of RAC cell 10. Liner 24 and cover 25 may be single layer or multiple layer sheets. Moreover, the layers can be of air and/or liquid permeable materials such as geotextile materials so long as at least one layer is an essentially air and gas impervious material layer.

FIG. 10 is a close-up side cutaway view of an edge of a RAC cell 10 that includes a piping penetration vault 42. In FIG. 10, piping penetration vault 42 is installed in a partial trench 55 constructed at a perimeter 26 of RAC cell 10 such that the top of vault vertical wall 44 is at grade 17. The vault trough 47 can be filled with compostable material 30 or, with soil or some other medium such as clay or gravel to protect the piping located in trough 47. Placing piping penetration vault 42 at an edge of RAC cell 10 as shown in FIG. 10 provides for a seamless transition between RAC cell 10 and the top edges of pit 20. FIG. 10 also includes an anchor trench 38. Anchor trench 38 functions to hold liner 24 in-place and to prevent it from sliding down the sidewalls.

FIG. 11A is a cross-section view of yet another in-situ RAC cell embodiment of this invention and FIG. 11B is a close-up cross section view of an anchor trench associated with the RAC cell of FIG. 11A. FIG. 11B in particular shows details regarding the retaining of and sealing of liner 24 in anchor trench 38. The liner shown in FIG. 11B is a multiple layered liner including liner 24 such as an HDPE liner that lines the bottom and sides of pit 20. Liner 24 is in turn covered by geotextile liner 72 which forms the top layer of liner 24. The top of RAC cell 10 includes cover 25 that in turn is covered with a flexible membrane liner 79. In FIG. 11B, the two cover layers are welded to the bottom layers at weld 66 located on the cell side of anchor trench 38. Cover 2, solid liner 70 and flexible membrane liner 79 enter anchor trench 38 such that the edge of flexible membrane liner 79 is located in the anchor trench. The edge 57 of liner 24 and the edge 58 of cover 25 emerge from anchor trench 38 where the edges are welded together by weld 67. Locating the liners in anchor trench 38 allows the liners to firmly held in place around the perimeter of cell 10. Anchor trench 38 may be offset from pit 20 or anchor trench may be formed around the top perimeter of pit 20 as shown in FIGS. 4A, 4B and 5.

Liner 24 and cover 25 are each include a perimeter edge 57 and 58 respectively. Liner 24 and cover 25 are sealed in anchor trench 38 by locating perimeter edges 57 and 58 in anchor trench 38 such that edges 57 and 58 lie entirely in anchor trench 38 or such that edges 57 and 58 lie beyond anchor trench 38 in relation to cell 10 as shown in FIG. 11B. Anchor trench 38 is then filled with soil, gravel, clay or some other material to secure perimeter edges 57 and 58 and thereby liner 24 and cover 25 in place and to seal RAC cell 10.

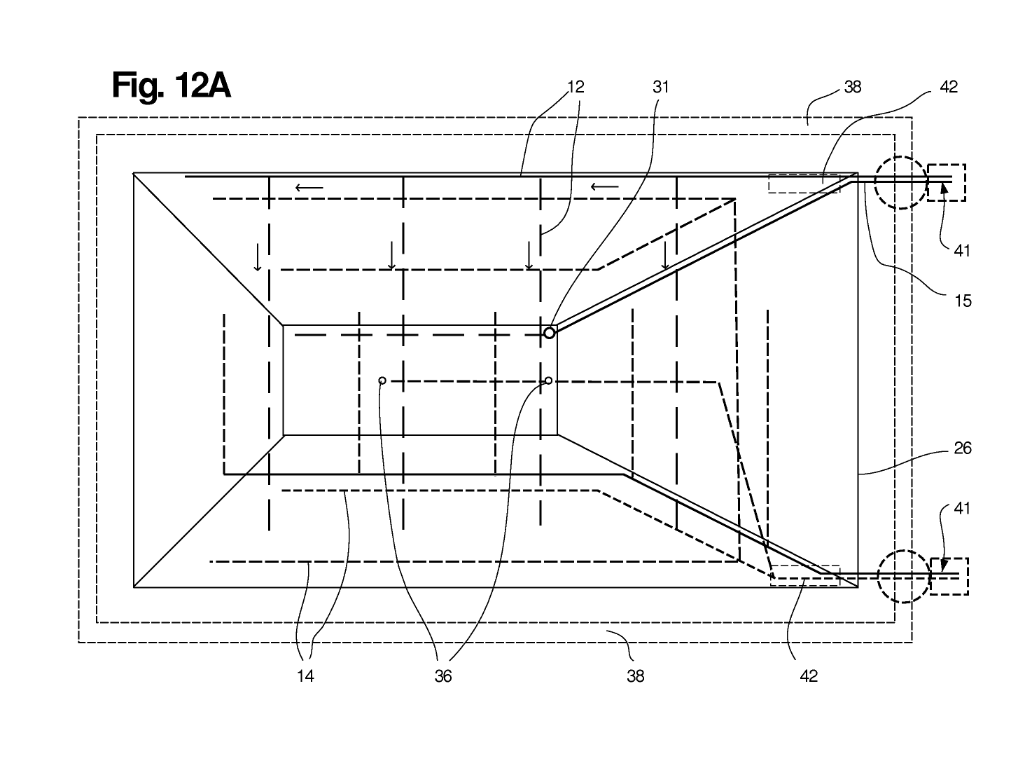

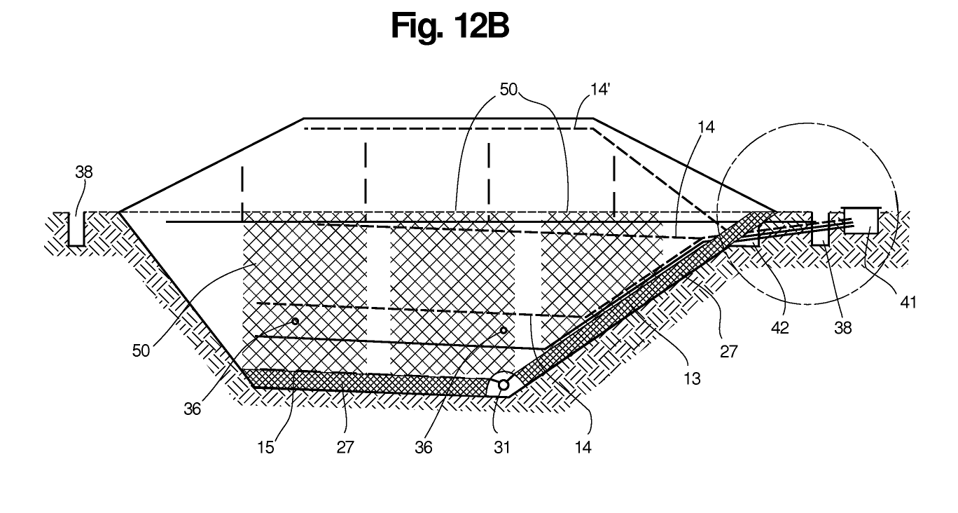

FIGS. 12A and 12B are plan and section views of yet another in-situ RAC cell embodiment of this invention that show further details of an alternative piping embodiments. In addition, the RAC cells of FIGS. 12A and 12B include an anchor trench 38 surrounding the perimeter of RAC cell 10, several piping vaults 42 as well as several piping pits 41. Piping pits 41 are useful for gaining access to important pipe fittings and they also provide a location to place monitoring instruments.

The piping used in and around the RAC cell and composter complexes of this invention may be any type of piping useful in landfill or composter applications. While the piping can be metal piping, it is preferred that the piping is plastic piping because of its price and ease of installation. Examples of useful plastic piping include, but are not limited to, PVC piping and HDPE piping. The piping used in RAC cell 10 will generally have diameter ranging from 2 inches to about 8 inches with diameters of 3 to 4 inches being preferred.

The piping that lies outside of RAC cell 10 will be solid piping. The piping installed inside RAC cell 10 can be solid piping or it can be perforated piping depending upon the piping application. For example, the gas removal piping will typically include many perforations or perforated sections to remove fermentation gasses from cell 10 in a manner that minimizes the pressure drop across the piping during vacuum gas recovery. The type of piping used and locations of perforations or pipe openings within the composter is well within the knowledge of one skilled in the art.

During normal operations, the quality of gas from each RAC cell is monitored—preferably automatically using sensors and a system that uploads readings to a monitoring location remote to the cells and activates alarms as necessary. Typical monitoring includes off gas methane level, balance gas, pH, gas flow, pressure and temperature. Additionally, hydrogen sulfide is sometimes monitored. Note the system can be monitored manually in the case of automation failure or in special circumstances.

The fermentation end of life is reached based on gas recovery and the gas curve. Once the gas curve has diminishing returns or looses temperature necessary for anaerobic digestion, the anaerobic fermentation is terminated by aeration and off gassing to the biofilter. Once the amount of methane in the off gas is reduced to a safe level, RAC cell dewatering also takes place through the sump. When the off gas shows greater than 5% oxygen in concentration and the odors are reduced, the cover can be removed. The RAC cell product—called digestate, is processed as noted below.

The composter embodiments of this invention may be prepared in accordance with one or more of the steps discussed below. An initial step can be a shredding and mixing stage. In the shredding and mixing stage, selected compostable material such as food and organics materials are source separated, sized by shredding if necessary, and then optionally mixed with other compostable materials such as an equal volume of shredded yard waste or woodchips to form a compostable mixture. If not loaded immediately into the RAC cell, the compostable material or compostable mixture is staged and odors and vectors are minimized by placing a layer of yard waste or compost over the pile until loading into the RAC cell is complete. The staged material may also be covered with a tarp. In some cases an alkaline material such as lime is added to the mixture.

Next, the compostable material or mixture is charged into the RAC. When operations are ready to charge a new RAC cell or to recharge a previously used RAC cell or pod, a seed material (digestate) from a recently finished cell is preferably mixed in a ratio of above 0% to 50% by volume with the compostable material or compostable mixture previously described to form a seeded compostable mixture. This seeding step decreases lag time in the anaerobic step and prevents a prolonged acid stage in the digestion process. In some cases leachate from an earlier digested cell is added instead or in combination with digestate to form the seeded compostable mixture. The use of leachate as a seed material is especially effective during warm weather periods and when the incoming waste materials include significant amounts of organisms that promote fermentation. This might include various manures, primary sludges and grease pit waste.

The seeded compostable mixture is loaded into the next open RAC cell which has its temporary plastic cover (such as 20 mil scrim) removed for loading. As loading of the RAC cell with the seeded compostable mixture continues, the cover is alternatively removed and replaced until the cell is full. Moreover, during RAC cell loading, a light vacuum may optionally be applied to the material in the partially filled cell using vacuum piping located at the cell bottom in order to prevent odors and VOC's from emanating from the partially filled cell. The gasses and odor bodies removed by vacuum are directed to a compost bio-filter adjacent to the cell. The seeded compostable material in the partially constructed cell is typically anoxic at this stage and is not producing significant methane.

The different piping systems discussed above will be added to RAC cell either before, during or after the compostable mixture is added to the cell. Generally, aeration system piping and leachate removal piping will be placed at or near the bottom of the cell pit before compostable material is added to the pit. The leachate injection piping can be added to the cell as vertically spaced planar piping manifolds as the compostable material is added to the pit. The gas extraction piping can be added to the cell as discussed above, as a plurality of vertically spaced planar piping manifolds or in any other manner known in the art including as vertical gas extraction wells.

Table 1 illustrates the impact of varying ratios of virgin compostable materials to recycled compostable materials in the seeded compostable material on fermentation cycle time;

TABLE 1
Cycle time vs. mix ratio

| Cycle Time | % New Material added | % Recycled Digestate |
|---|---|---|
| Days | Digester | Ratio* |
| 20-60 | 50 | 50 |
| 61-120 | 40-50 | 50-60 |
| 121-200 | 30-40 | 60-70 |
| 201-300 | 20-40 | 60-80 |
| >300 | 10-30 | 70-90 |

*Note, if more than 10% biological sludge's or manure is added to the digester the recycle (digestate) ratio may be adjusted by as much as 100%, especially in long retention times.

Once filled with seeded compostable material, the RAC cell is ready to be sealed. Before the RAC cell is sealed piping is placed on the top of the mixed feed and the RAC is sealed with a cover (typically 40 mil LLDPE) that is secured either by plastic welding or by securing in an adjacent anchor trench backfilled with soil, clay or some similar seal material. The anaerobic (without air) phase of fermentation soon begins. Alternatively the cell is made airtight with a prefabricated cover. Each RAC cell is intended to be air tight and vacuum aids in removing anaerobic fermentation product gases.

Once an RAC cell is filled with compostable material and the cover is attached and sealed, the individual RAC cell reaches anaerobic fermentation condition quickly. Once sealed, the vacuum system to the biofilter is turned off and RAC cell off gas pressure and gas quality is monitored. As soon as the gas is oxygen free, vacuum can be applied to the methane removal system. Converting the RAC cell to anaerobic conditions can be accelerated by several methods including by using an optional air blow (aeration) step. The aeration step allows for transition of the compostable material out of the acid phase quickly thus preserving the fermentables for energy producing gas. In order to raise the internal waste temperature to an operating range between 40° C. and 75° C., short term air injection may sometimes be useful in certain circumstances where the feedstock may be particularly acidic in nature (citrus, tomato, or fruit dominated) and where ambient temperatures are below 70° F. This aeration step rapidly digests volatile organic acids and raises the pH to above 6.5. The air can be injected into the seeded compostable material in the RAC cell using any piping that is in place such as the aeration/vacuum piping located at the bottom of the cell or by using the leachate injection piping that is optionally placed throughout the seeded compostable material. During this optional step, the gas extraction system withdraws the exhaust gas products from the RAC cell and preferably directs them to a biofilter for treatment.

Once anaerobic fermentation conditions are reached, the gas extraction piping and gas extraction system begins removing the gaseous anaerobic fermentation products from the RAC cell, preferably using a vacuum pump to extract the useful gases. Moisture, in the form of liquid removed from other anaerobic RAC cell cells can be added to a newly operational RAC cell to increase the availability of methaneogenic seed. Additionally, the moisture content and pH of the new RAC cell is monitored at start-up and the cell pH adjusted to prevent undesirable acid phase conditions. Methane is expected to be present in the extracted gas at levels of approximately 40-75%. The extracted gases can be used for many purposes including for transportation fuel or for energy production. Because the RAC cell is completely sealed, no methane emissions from the fermentation process is anticipated. Estimated total fermentation time (residence time) of a single RAC cell is expected to as short as 25 days and as long as 270 days or more. Variance in residence time will be based on the digestion rate of variable feedstock and climate influence (colder, slower) on the rate of gas production.

Once the selected anaerobic fermentation end point is reached the RAC cell can be opened and the solid digestate removed or the RAC cell is operated in a maturation step. For example, in one embodiment, the anaerobic end point is reached when the gas generation rate is diminished significantly—e.g. to below 50% of original at which point the anaerobic phase is terminated by adding air to the system. However, because the RAC cells of this invention are so economical to install and operate, the anaerobic fermentation step can be allowed to continue until the methane product rate is significantly below the start-up methane product rate. It is expected that the RAC cells of this invention will be able to be operated at methane product rates as low as 25% or less of the start-up methane product rates. The anaerobic fermentation end point can alternatively be identified when the cell temperature reaches a certain point or by any other means known on the art for measuring anaerobic fermentation progress.

At the selected fermentation end point, air can be added to the RAC cell by blowing air through the vacuum piping installed at the bottom of the RAC cell. In addition to ending methane generation, stopping anaerobic fermentation begins the digestate maturation step, which will typically last 2-4 weeks. During digestate maturation, most free liquids are removed from the cell by leachate removal piping and sent to a storage tank and/or used as seed in another developing RAC cell. Upon completion of digestate maturation or once the RAC cell becomes aerobic, the cover is removed and the digestate is recovered for reuse as charge material or mixed and amended for a compost product. In one embodiment, the digestate is removed and at least part of the digestate is mixed with new incoming compostable material that is rough shredded. The shredded compostable material may include, for example, yard waste, manure, sludges, wood, pallets, brush, food waste, and cellulosic materials like cardboard. Mix ratios may vary based on the amount of moisture and particle size of the compostable material components. In the dry season more food waste is added to the reactor and conversely in the wet season or when yard waste is readily available, the amounts of green grass and wood chip ratio is changed. The amount of digestate mixed with the new material also varies. More previously treated material is mixed if a shorter cycle is needed, <60 days, and this ratio is modified up to a 12 month residence time. In some cases waste heat in the form of steam is added to the pit or lechate tank in order to maintain or increase fermentation rates.

During the entire process, sensors can be used to monitor and control the process. Temperature monitoring of all the RAC cells is preferably continuous. In the event out of range temperatures are observed in an operating RAC cell, liquid is added through leachate injection piping or any other available piping in order to quench and cool the fermentation reaction. The RAC cell design—which preferably includes berms—allows for flooding of the RAC cell up to the height of the side walls and direct recirculation of liquids. Liquid levels are controlled by the sump collection system and recirculation piping.

The RAC cell cells useful in the present invention can vary from a 400 ton capacity to 4,000 ton capacity at a placement density of 1400 to 1600 lbs/cubic yard. This variable capacity requires that a vacuum is applied to the material in the digester after it is partially filled. This action removes odors and other volatile gases for treatment in a compost based biofilter. Furthermore, a temporary cover is provided for daily covering of the digester to further aid in odor and volatiles capture. The application of the vacuum to the shredded material causes the material to start to aerobically compost. This action raises the temperature to temperatures >120° F. and as much as 160° F. As an alternative, aeration is initially supplied to the mass and excess air is treated in the biofilter. This activity also induces heterotrophic degradation of the mass yielding heat.

The in-situ RAC cells of the invention are useful for generating methane gas that is useful for producing energy from food waste and yard waste previously landfilled or aerobically composted. The in-situ RAC cell can be located on a landfill, a landfill buffer area, a transfer station, a composting yard, a closed landfill or at a food manufacturing facility. Residual solids in the digester also produce a product called digestate, this has various horticultural uses. The energy component is the result of anaerobic fermentation producing high quality methane. The in-situ design allows for fire suppression by complete aqueous filling of 80% to 90% of the reactor if needed. The invention allows for easy sourcing of commercial wastes (like grocery wastes) that includes large amounts of cardboard and wax covered cardboard. It reduces fuel use by hauling companies by not changing the delivery location at existing solid waste facilities in many instances.

What is claimed is:

1. A method for anaerobically fermenting compostable materials in-situ comprising the steps of:
preparing compostable material;
preparing a cell for holding the compostable material the cell including a pit constructed in a section of ground, the pit including side walls, a bottom, an essentially impervious liner located in the pit such that the liner abuts the pit side walls and bottom to form a lined pit;
placing the prepared compostable material in the cell;
covering the cell with a cover to form an essentially gas tight anaerobic composter cell;
bringing the cell to fermentation conditions and operating the cell at anaerobic fermentation conditions sufficient to form digestate and anaerobic fermentation gasses;
collecting the anaerobic fermentation gasses using gas extraction piping located in the cell;
halting the anaerobic fermentation when a defined anaerobic fermentation end point is reached; and
opening the cell and removing the digestate to form an emptied cell.

2. The method of claim 1 wherein the cell further includes at least one pipe for injecting leachate into the compostable material and at least one pipe for removing aqueous materials that collect on the bottom of the lined pit from the composter.

3. The method of claim 1 wherein the compostable material is seeded with anaerobic microorganisms at a time selected from the group consisting of before placing the prepared compostable material in the cell, after placing the prepared compostable material in the cell, or both before and after placing the prepared compostable material in the cell.

4. The method of claim 1 wherein the pit bottom includes aeration piping and wherein a vacuum is applied intermittently or continuously to the prepared compostable material as the pit is being filled with compostable material.

5. The method of claim 1 wherein the essentially gas tight anaerobic composter cell is converted to anaerobic conditions by blowing air into the prepared compostable material until the cell fermentation off gas is essentially oxygen free.

6. The method of claim 1 wherein the liner and cover are associated with one another to form an essentially gas tight anaerobic composter cell.

7. The method of claim 6 wherein the liner and cover are sealed by welding to each other.

8. The method of claim 6 wherein an anchor trench is formed around the perimeter of the cell and wherein the perimeter of the cover and the perimeter of the liner are both located in the anchor trench and thereafter filling the trench with a seal material to form an essentially gas tight anaerobic composter cell.

9. The method of claim 1 wherein the digestate is allowed to mature for from 1 day to about four weeks or more.

10. The method of claim 1 wherein a new batch of compostable material is placed in the emptied cells and the method is repeated.

11. The method of claim 1 wherein the prepared compostable material is a mixture of digestate and shredded compostable material.

12. The method of claim 11 wherein the ratio of digestate to shredded compostable material ranges from about 10:1 to about 1:10.
